(19) United States
(12) Patent Application Publication
(10) Pub. No.: US 2024/0099743 A1
(43) Pub. Date: Mar. 28, 2024

(54) FOETAL EXTRACTION DEVICE

(71) Applicant: TECHNISCHE UNIVERSITEIT ENDHOVEN, EINDHOVEN (NL)

(72) Inventors: Rachel Francisca Angelica Maria Loop, EINDHOVEN (NL); Franciscus Leonardus Marie DELBRESSINE, EINDHOVEN (NL); Franciscus Nicolaas VAN DE VOSSE, EINDHOVEN (NL); Swan Gie OEI, EINDHOVEN (NL)

(21) Appl. No.: 17/767,588

(22) PCT Filed: Oct. 12, 2020
(86) PCT No.: PCT/EP2020/078568

§ 371 Date: Apr. 8, 2022

Related U.S. Application Data
(60) Provisional application No. 62/914,429, filed on Oct. 12, 2019.

(51) Int. Cl. A61B 17/44

(52) U.S. Cl. CPC A61B 17/44

(57) ABSTRACT

A phoetal (i.e., fetal) extraction device including a frame configured to hold a biobag for receiving a baby, the frame having a distal end for insertion into a vagina, a longitudinal axis aligned with a insertion direction, and a radial direction. The frame includes frame members having distal end configured for insertion and proximal ends attached to a connecting element. The frame members extend away from the connecting elements and are circumferentially spaced apart around the longitudinal axis. The frame members are expandable in the radial direction such that a radial position of radial orientation of the frame members is adjustable relative to the connecting element. A kit of parts including at least the frame and the biobag.

### FIELD OF THE INVENTION

The invention relates to a foetal extraction device, a biobag for use with such a foetal extraction device, a foetal extraction device kit-of-parts, as well as a method for foetal extraction using such a foetal extraction device.

### BACKGROUND OF THE INVENTION

Preterm children born as early as 24 weeks of gestation are underdeveloped when they take their first breath. Preterm birth makes it especially hard on the lungs as they are not yet capable of supporting the whole body with oxygen. Ventilation is often used, but sometimes harms as much as it benefits. Due to the above, preterm children have a very low survival rate and if they survive they are often impaired due to brain damage or other developmental consequences.

### SUMMARY OF THE INVENTION

According to the invention, a foetal extraction device is provided, comprising:
a frame configured for holding a biobag inside the frame, the biobag being arranged for receiving a baby, having a distal end arranged to be inserted into a vagina, with a longitudinal axis (X) aligned with an insertion direction (I), and a radial direction (R), the frame comprising a plurality of frame members with distal ends arranged to be inserted into the vagina and proximal ends attached to a connecting element, the frame members extending away from the connecting element in the insertion direction and being circumferentially spaced-apart around the longitudinal axis of the frame, wherein the frame members are expandable in the radial direction, and wherein the radial position or the radial orientation of the frame members with respect to the connecting element is adjustable.

The above-described frame can be advantageously used to insert, position and hold a biobag in the vagina for receiving the neonate/baby.

The biobag catches the neonate when leaving the vagina and the foetal extraction device with the abovementioned frame guides the neonate safely into the biobag. The biobag can then be sealed in such way that the umbilical cord of the neonate is not clamped off. Then, the frame is removed from the birth channel and afterwards the biobag containing the neonate is removed from the birth channel. The neonate can then be connected appropriately to an artificial womb device.

Thus, the invention provides technology to enable transfer of preterm babies into a safe environment where they can mature further without leaving the amniotic fluid and starting to breathe. Once the development of the baby is sufficient they can then be exposed to the real world. Thus, the foetal extraction device according to the invention reduces the risk for complications in later pregnancies which could be due to incisions in the womb by a caesarean section in present procedures reducing or eliminating.

An embodiment relates to an aforementioned foetal extraction device, wherein, when viewed along the longitudinal axis (X), the frame members have a first frame member portion and a second frame member portion, and wherein an interior side of the first frame member portion is concave and an interior side of the second frame member portion is convex. The concave portion can advantageously be used to hold the biobag (with the head of the neonate), whereas the convex portion can be easily inserted in the vagina to force the biobag to stay open and to receive and guide the head of the baby into the biobag.

An embodiment relates to an aforementioned foetal extraction device, wherein the frame has a frame length L from the proximal ends to the distal ends of the frame members and the first concave frame member portion transitions into the second convex frame member portion at a transition position (T) located at about 0.6 L-0.8 L from the proximal ends of the frame members. Thus, an optimal ratio is achieved between the concave and convex portions, with the transition position located approximately near the vagina entrance.

Therein, the frame members preferably have an S-shape, which shape ensures optimal expandability and deformability/bendability.

An embodiment relates to an aforementioned foetal extraction device, wherein the frame members are circumferentially spaced-apart around the longitudinal axis of the frame at regular intervals.

An embodiment relates to an aforementioned foetal extraction device, wherein each of the plurality of frame members comprises a group of individual, parallel frame elements extending away from the connecting element in the insertion direction, such as groups of 2, 3, 4, 5, 6-10 or more individual frame elements. The individual frame elements can deform/expand/bend with respect to each other to allow the frame members to optimally engage and conform to the contours of the head of the baby.

An embodiment relates to an aforementioned foetal extraction device, wherein the distal ends of the frame members are provided with rounded knobs to prevent damage to the head of the baby.

An embodiment relates to an aforementioned foetal extraction device, wherein the connecting element comprises a through-hole extending through the connecting element along the longitudinal axis (X), wherein the connecting element preferably has a ring-shape. Thus, tubes, cords, et cetera can be connected to the biobag and operated via the through-hole in the connecting element.

An embodiment relates to an aforementioned foetal extraction device, wherein, when the frame members are arranged in a fully expanded state, a maximum diameter (Dₘₐₓ) of an opening radially enclosed between the distal ends of the frame members is about 6-10 cm, preferably 8 cm.

An embodiment relates to an aforementioned foetal extraction device, wherein the frame has a frame length L from the proximal ends to the distal ends of the frame members of 30-50 cm, preferably 40 cm to ensure portability and optimal handling.

An embodiment relates to an aforementioned foetal extraction device, wherein one or more of the frame members have an elliptical cross-section, wherein a short axis of the elliptical cross-section is aligned with the radial direction. Thus, when the distal ends of the frame members engage the baby's head, proper engagement/contact is achieved between the frame members and the baby's head, without damaging the baby's head.

An embodiment relates to an aforementioned foetal extraction device, wherein, in the radial direction (R), a bending stiffness at the proximal end of one or more frame members of the plurality of frame members is higher than a bending stiffness at the distal end of the one or more frame members of the plurality of frame members. By allowing the distal ends of the one or more frame members to be more flexible than the proximal ends, easier engagement with the baby's head can be achieved without wounding the baby's head (or the mother).

An embodiment relates to an aforementioned foetal extraction device, wherein comprising:
a handle, extending along the longitudinal axis (X), the handle having a proximal end and a distal end connected to the connecting element.

The above handle allows the frame to be pushed and pulled into and out of the vagina as necessary.

An embodiment relates to an aforementioned foetal extraction device, wherein the handle comprises a through-hole extending through the handle along the longitudinal axis (X), wherein the handle through-hole and the connecting element through-hole preferably form a single, continuous through-hole. Again, this allows tubes, cords, et cetera to be connected to the biobag and operated via the through-hole in the handle.

An embodiment relates to an aforementioned foetal extraction device, comprising:
an adjustment element slideably arranged over the plurality of frame members along the longitudinal axis (X), the adjustment element being configured for adjusting the radial position or the radial orientation of the frame members when sliding the adjustment element over the frame members along the longitudinal axis (X).

Thus, the radial expansion of the frame members can be adjusted.

Preferably, the adjustment element is a tubular element.

Furthermore, one or more transitions between one or more adjacent parts of the foetal extraction device are preferably smooth, in particular the one or more transitions between the distal ends of the frame members and the rounded knobs. Thus, accumulation of pathogens, such as bacteria, between one or more adjacent parts of the foetal extraction device can be prevented and hygiene is promoted.

An embodiment relates to an aforementioned foetal extraction device, comprising:
a biobag arranged inside the frame for capturing a baby during delivery, wherein an open end of the biobag is arranged near the distal ends of the frame members.

As explained before, the frame can be advantageously used to insert, position and hold the biobag in the vagina, wherein the biobag forms the safe environment for the neonate.

An embodiment relates to an aforementioned foetal extraction device, wherein a vacuum (e.g. silicone) ring is provided circumferentially around the open end of the biobag, to cause the biobag to push open against the borders of the birth channel, sealing the environment between the opening of the bag and the cervix.

An embodiment relates to an aforementioned foetal extraction device, comprising a cord provided circumferentially around the open end of the biobag for closing the open end of the biobag. E.g. while the bag with the baby is supported in the hand of medics, the cord can be pulled to decrease the size of the opening of the bag.

An embodiment relates to an aforementioned foetal extraction device, wherein the biobag is provided with one or more tubes for supplying one or more fluids to the biobag, such as disinfectant or artificial amniotic fluid.

An embodiment relates to an aforementioned foetal extraction device, wherein an outside or outer surface of the biobag is provided with one or more sensors, such as temperature sensors, heartbeat sensors (or in general sensors for monitoring vital signs), or sensors for monitoring contamination of the amniotic fluid inside the biobag, and/or actuators, such as tactile, visual or sonic actuators. Thus, the baby inside the biobag can be monitored from the outside, especially after delivery, and/or inputs can be provided to the baby inside the biobag e.g. resembling the touch of the parents (tactile), resembling audio-visual conditions in the womb, et cetera.

Preferably, the biobag is configured in such a way, that the inside of the biobag can be inspected visually and/or via ultrasound and/or via infrared sensing, in particular after delivery of the baby, i.e. when the baby is inside the biobag.

As stated before, another aspect of the invention relates to a biobag for capturing a baby during delivery, configured for being arranged inside the frame of an aforementioned foetal extraction device, wherein an open end of the biobag is configured for being arranged near the distal ends of the frame members.

Another aspect of the invention relates to a foetal extraction device kit-of-parts, comprising:
an aforementioned foetal extraction device comprising a frame,
a handle configured for extending along the longitudinal axis (X), the handle having a proximal end and a distal end connectable to the connecting element,
an adjustment element configured for being slideably arranged over the plurality of frame members along the longitudinal axis (X), the adjustment element being configured for adjusting the radial position or the radial orientation of the frame members when sliding the adjustment element over the frame members along the longitudinal axis (X), and
a biobag configured for being arranged inside the frame for capturing a baby during delivery, wherein an open end of the biobag is configured for being arranged near the distal ends of the frame members.

Another aspect of the invention relates to a method for foetal extraction using an aforementioned foetal extraction device, comprising the steps of:
at a first degree of dilation of a vagina of a mother that is to deliver a baby, preferably at around 5 centimetres of dilation: inserting the distal end of the frame into a vagina of the mother, preferably under ultrasound guidance,
pressing the distal end of the frame against a head of the baby,
receiving the baby in a biobag arranged inside the frame for capturing the baby, wherein an open end of the biobag is arranged near the distal ends of the frame members.

### BRIEF DESCRIPTION OF THE DRAWINGS

The invention will next be explained by means of the accompanying drawings and description of the figures. Therein:

FIG. 1 shows an embodiment of the foetal extraction device;

FIG. 2 shows frame behaviour with the handle moved upwards, showing the frame members after movement and before movement;

FIG. 3 shows frame behaviour with the frame members in a straightened position in an expanded birth channel and in a former, bent position, wherein the perinate moves through the birth channel;

FIG. 4 shows frame behaviour with the perinate out of the mother's body, situated in the expanded belly of the frame, as well as the previous position of the frame members as shown in FIG. 2, and the birth channel returned to normal shape;

FIG. 5 shows an embodiment of the (silicone) biobag;

FIG. 6 shows the biobag positioned in the frame with a portion of the biobag folded around the distal ends of the frame members;

FIG. 7 shows a close up of the biobag as shown in FIG. 6;

FIG. 8 shows a position of the biobag and frame after opening of the vacuum ring;

FIGS. 9A-9C show the retrieval of a neonate using the frame and biobag; and

FIG. 10 shows the biobag arranged inside the frame, with the open end of the biobag in an unfolded state.

### DETAILED DESCRIPTION

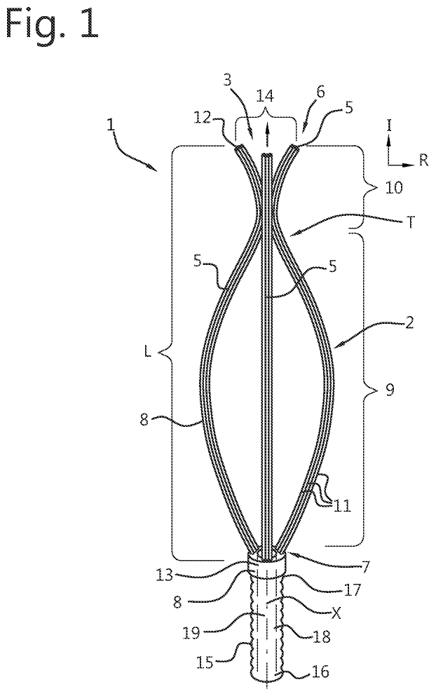

As shown in FIG. 1, an embodiment of the foetal extraction device 1 comprises a frame 2 configured for holding a biobag inside the frame 2, the biobag being arranged for receiving a baby. The frame 2 has a distal end 3 arranged to be inserted into a vagina, with a longitudinal axis X aligned with an insertion direction I, and a radial direction R. The frame 2 comprises a plurality of frame members 5 with distal ends 6 arranged to be inserted into the vagina, and proximal ends 7 attached to a connecting element 8. The frame members 5 extend away from the connecting element 8 in the insertion direction I and are circumferentially spaced-apart around the longitudinal axis X of the frame. The frame members 5 are expandable, i.e. bendable or elastically deformable, in the radial direction R, wherein the radial position or the radial orientation of the frame members 5 with respect to the connecting element 8 is adjustable. The frame 2 may have e.g. three frame members 5 in the form of arms 5 and is preferably manufactured out of sterilisable plastic. The arms 5 are curved, preferably have an S-shape and are circumferentially equally distributed on the connecting element 8. When viewed along the longitudinal axis X, the frame members 5 essentially have a first frame member portion 9 and a second frame member portion 10, wherein an interior side of the first frame member portion 9 is concave and an interior side of the second frame member portion 10 is convex. Each arm 5 has a curvature that has a top and a belly bent in opposite directions (thus resembling an S-shape). Preferably, the frame 2 has a frame length L from the proximal ends 7 to the distal ends 6 of the frame members and the first concave frame member portion 9 transitions into the second convex frame member portion 10 at a transition position T located at about 0.6 L-0.8 L from the proximal ends 7 of the frame members 5.

The distal ends 6 of the arms 5 form a funnel-like structure. At the edges of this funnel-like structure individual spikes 11 are shaped into protective knobs 12.

The handle 15 as shown is hollow (has a through-hole 18) over its full length and additionally a through-hole 13 is provided in the middle of the connecting element 8 with an exemplary diameter of about 1 cm, i.e. the handle 15 and the connecting element 8 comprise a continuous through-hole 19 extending through the handle 15 and connecting element 8 along the longitudinal axis X. The connecting element 8 preferably has a ring-shape. The frame 2 is approximately 60 cm tall, with a 10 cm handle, and 50 cm arm length, e.g. subdivided into a 2 cm connecting element length, 35 cm belly length and 8 cm top length.

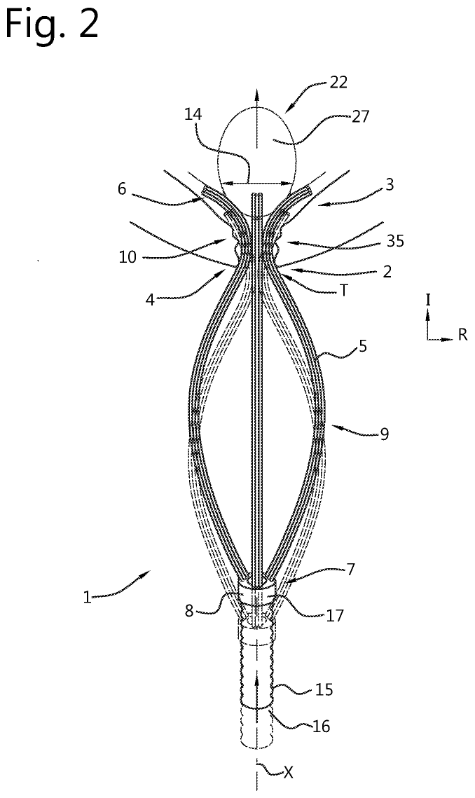

Referring to FIG. 2, at approximately five centimetres dilation only the top (distal end 3) of the frame 2 is inserted into the mother under ultrasound supervision. The handle 15 is lightly pressed or pushed in the insertion direction I so the funnel expands slightly against the head 27 of the baby 22. The perinate 22 is represented as an ellipsoid in FIG. 2. The shape of the funnel is designed to avoid damage to the head 27 of the neonate 22. Even when born in breech position the shape of the funnel is intended to avoid damage to the neonate. The dashed lines in FIG. 2 show the former position of the frame 2 without funnel expansion around the perinate 22. FIG. 2 shows the later position with expansion.

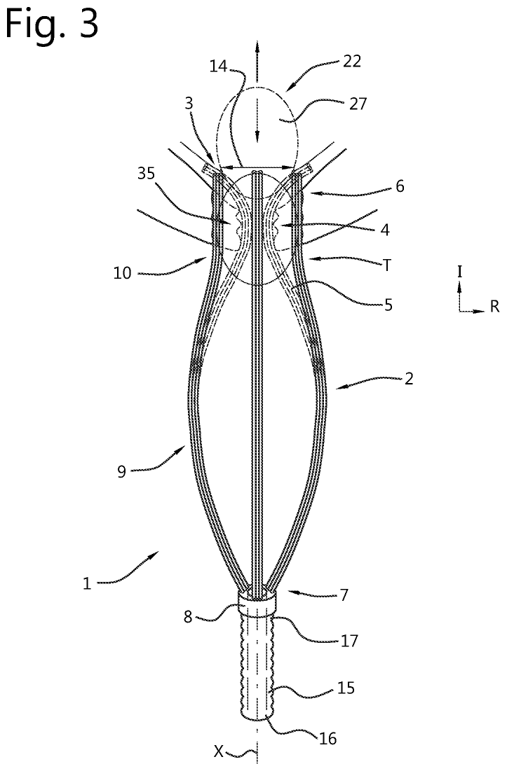

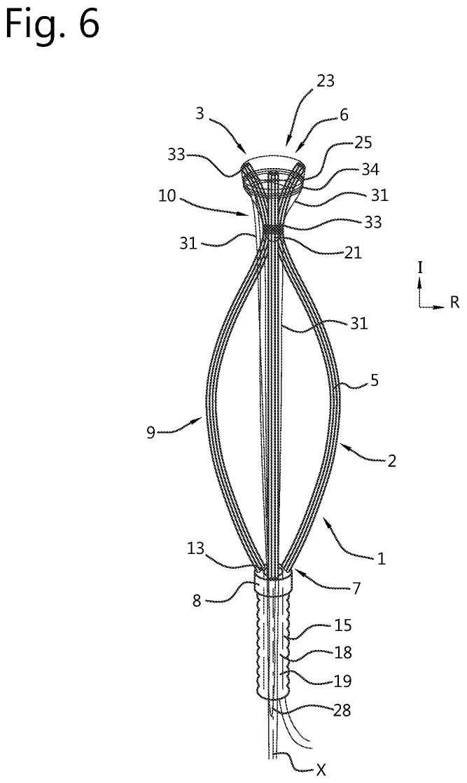

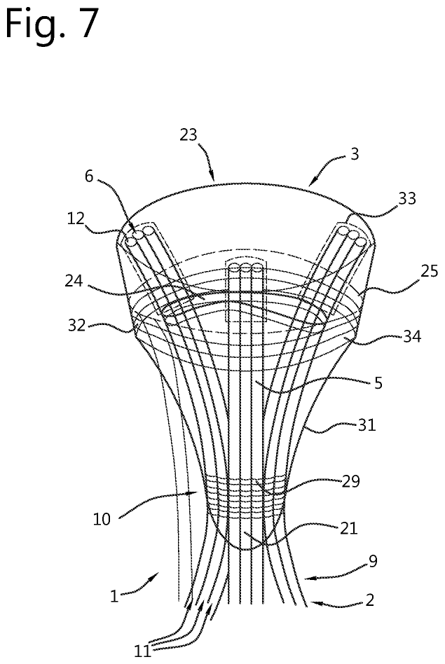

With the frame 2 in place the baby 22 will be safely guided out of the womb. The baby 22 will slide through the frame 2 on its own because the cervix 35 retracts. If needed suction can be applied by generating a lower pressure in the biobag to catch the neonate 22 in the biobag (please refer to FIGS. 6-10 for the location/arrangement of the biobag 21 with respect to the frame 2). While the perinate 22 presses on the inside of the funnel-shape and pushes the arms 5 of the frame 2 against the tissue of the mother on the outside it causes the distal end 3 to flatten out and the birth channel 35 to expand (see FIG. 3). This way the curved shape of the distal ends 6 of the arms 5 will give way for the perinate 22 and guide it along a direct path towards the centre of the biobag.

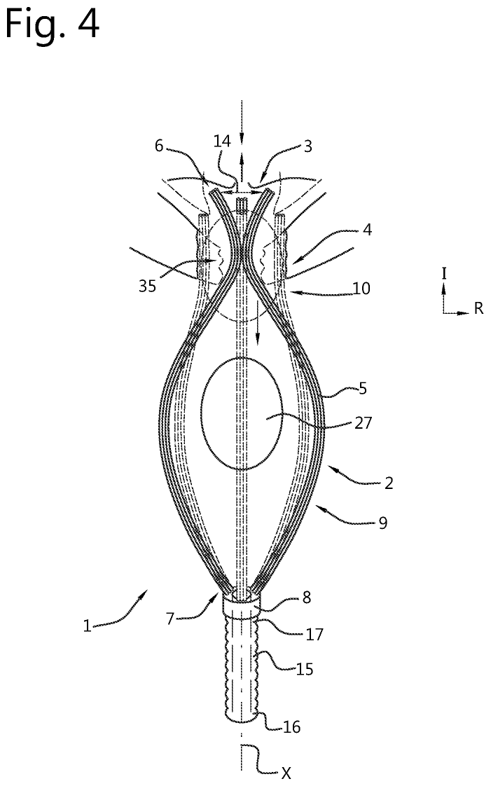

Once the baby/perinate 22 has moved through the birth channel 35 towards the middle of the frame 2 (and thus the middle of the biobag 21 (as described in FIGS. 6-10) as shown in FIG. 4, the frame 2 will restore its shape from the flattened position (as shown in FIG. 4) due to the elastic behaviour of the material. The belly of the frame's 2 arms 5 will expand if necessary, in line with the pressure on the inside (see FIG. 4). When the frame 2 restores it shape, so will the vaginal tissue while the cervix 35 is retracted as normal after birth (FIG. 4).

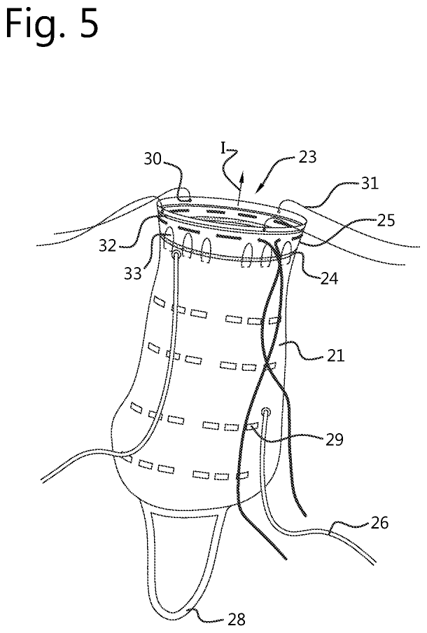

Referring now to FIG. 5, in an embodiment, the biobag 21 is made out of a flexible material, for example sterilisable silicone. The biobag 21 has a handle 28 at the bottom end thereof and has attached tubes 26 for the inflow and outflow of disinfectants and artificial amniotic fluid. A long string (approx. two meters) 25 is woven around the opening 23 of the biobag 21 like a purse string suture. Above the woven string 25 is a circumferentially placed balloon 32. On the very edge of the biobag 21 are four connection points 30 that attach cords 31 to the biobag 21. The biobag 21 is attached to the frame 2 the “spikes” (individual elements 11) inserted through the loops 29 and the knobs 12 ending in pockets 33. Underneath (and overlapping with) the pockets 33 is a rigid silicon vacuum ring 24.

Before insertion the biobag 21 and frame 2 are sterilized. Then the biobag 21 is attached to the inside of the frame (as shown in FIG. 6), with the spikes (individual elements 11) inserted through the loops 29 and the ends 6 encapsulated in the pockets 33. The lower part of the biobag 21 is not open yet, it still sticks together maintaining a lower than atmospheric pressure inside. The biobag 21 rests at the position where the frame 2 diameter is the smallest, in between the top 3 and the belly (i.e. concave portion 9 of the frame members 5. The rigid ring 24 remains in a folded, compressed position inside the frame 2, but the rim of the biobag 21 above the pockets 33, is folded backwards (34) over the edges 6 of the frame 2 with the cords 31 and the string 25 from the woven suture technique pointed towards the proximal end 7 of the frame 2, to remain easily accessible after insertion by guiding them through the hole 13 of the handle 28 or in between the arms (not shown). The balloon 32 is located at the bottom of the fold 34. The cords 31 attached to the edges of the frame 2 are guided through the loops 29 of the biobag 21 in opposite direction with respect to the frame 2. The tubes 26 rest loosely in between the spikes 11 of the frame 2 (not shown). The handle 28 is pulled through the opening 13 in the handle 15, far enough to come out at the bottom (i.e. the proximal end 16 of the handle 15), but not enough to pull the biobag 21 open (FIG. 6).

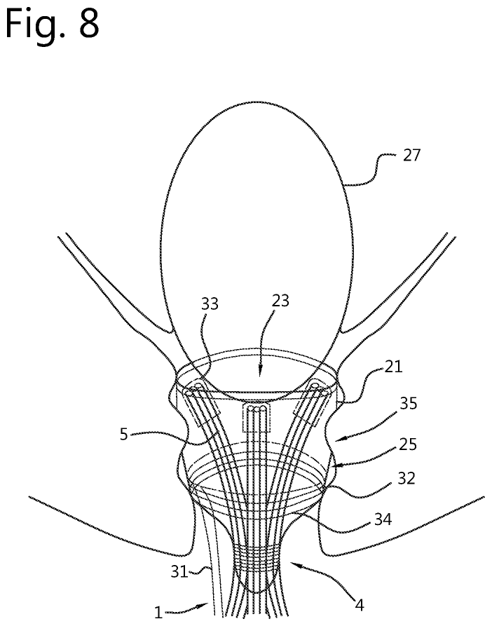

Now the frame 2 is ready for insertion. The top/distal end 3 is manually compressed and if necessary lube is administered, and then inserted into the vagina 4 where the frame 2 returns to the original shape (FIG. 2—dashed lined shape). At this point the birth channel 35 is sterilized by pumping disinfecting agents in through one of the tubes 26 (FIG. 5)—excess fluid flows out on the sides. Once this is done, the cords 31 are pulled to draw back the fold 34 slightly more to pull the vacuum ring 24 outwards over the edge/distal end 6 of the frame 2 to release it. The ring 24 is made out of a rigid silicone and wants to form back into its round shape, this will cause it to push open against the borders of the birth channel 35, sealing the environment between the opening 23 of the biobag 21 and the cervix 35 (FIG. 8).

Now the frame 2 is carefully pushed into position as shown in FIG. 2. The opening 23 of the biobag 21 can now be arranged directly around the baby/perinate's 22 exposed body. Artificial amniotic fluid is now pumped into the biobag 21 and excess air is pushed out. Once dilation is sufficient the infant will slide through the arms of the frame 2 into the biobag 21 on its own (FIG. 3), or by assistance of pulling the handle 28 (FIG. 6) of the biobag 21 which creates a negative pressure difference resulting in a suction force. When the handle 28 is pulled, the loops 29 will slide down, guided over the frame 2, forcing the bottom of the biobag 21 to open and increasing the volume. Because the volume of the biobag 21 increases while the environment is sealed it will create a suctioning effect on the exposed part of the perinate's 22 body. Since the gynaecologist is operating this action manually, he/she will have direct control over the exerted suction force. If the action is not sufficient to guide the perinate 22 out, the fluid can be pumped out, which shrinks the biobag 21, to repeat the action. If the seal is broken during the action, the cords 31 need to be pulled to reposition the vacuum ring 24 back into place and excess air needs to be pumped out before repeating the action.

Once the perinate 22 safely entered the biobag 21 and is positioned as in FIG. 4, the frame 2 can be removed (the seal of the vacuum ring 24 will keep the biobag 21 open and in place), while the biobag 21 with the baby 22 is supported in the hand of the medics, and the purse strings 25 can be pulled (FIG. 5) to decrease the size of the opening 23 of the biobag 21. The balloon 32 is now inflated with a liquid gel to seal the opening around the umbilical cord without clenching it.

Because the gel distributes itself due to the pressure exerted, it prevents occlusion of the veins in the cord. The seal of the vacuum ring 24 is broken when the purse strings 25 are pulled because it is forced inwards, decreasing its radius and making it fold. The handle 28 that is now completely free due to the removal of the frame 2, can now be used to pull the remaining part of the biobag 21 out of the birth channel 35. The freed umbilical cord can now be clamped and plugged onto the artificial womb.

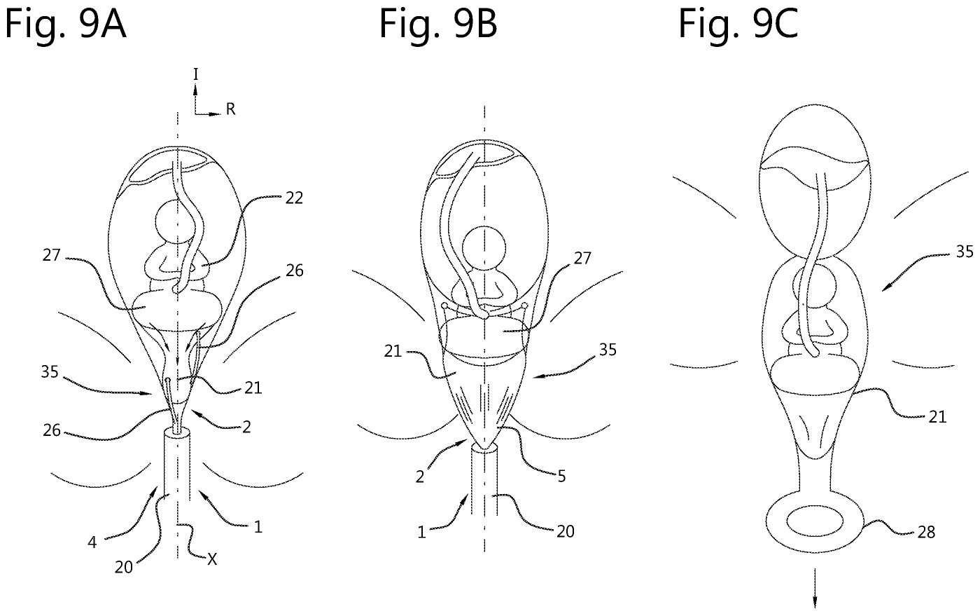

FIGS. 9A-9C furthermore show the frame 2 being inserted through a an adjustment element 20 in the form of a rod 20, and unfolding when the rod 20 is pulled back. The first stage of pulling back opens the biobag 21 enough for the birth channel 35 to be flushed, the next stage creates a vacuum, and in the last phase the frame 2 is fully released and the rod 20 removed. The unfolding pulls the biobag 21 open and due to the vacuum sucks the perinate 22 into the biobag 21. The sealing mechanism is closed behind the infant 22 and the frame 2 is removed. The biobag 21 is then gently pulled out of the birth channel 35.

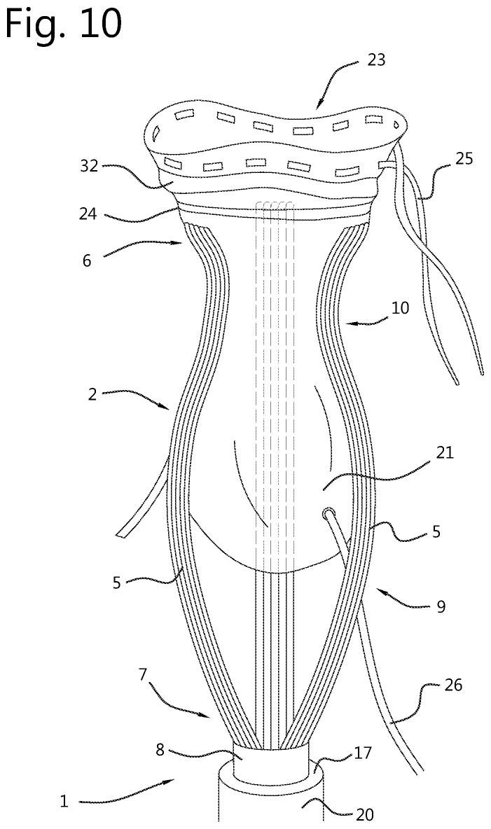

FIG. 10 shows another depiction of the frame 2 and the biobag 21. At the top the purse string 25 is visible that gets pulled for decreasing the size of the opening 23. Directly below the purse string 25 is the balloon 32 that will be inflated with gel around the umbilical cord. Thereunder, is the vacuum ring 24 that seals the disinfected birth channel 35 after it is flushed. Under the vacuum ring 24 are the folds or pockets 22 that slide over the frame 2 and keep the biobag 21 connected to the frame 2. There are tubes 26 attached for the inflow and outflow of disinfecting agents and artificial amniotic fluid on the silicone compartment that encloses the child 22. The rod or pipe 20 at the bottom of FIG. 10 can again be slid over the frame 2 to compress or expand (adjust) it in radial direction.

### LIST OF REFERENCE NUMERALS

1. Foetal extraction device
2. Frame
3. Distal end of frame
4. Vagina
5. Frame member
6. Distal end of frame member
7. Proximal end of frame member
8. Connecting element
9. First frame member portion
10. Second frame member portion
11. Individual frame element
12. Rounded knob
13. Through-hole in connecting element
14. Opening between distal ends of frame members
15. Handle
16. Proximal end of handle
17. Distal end of handle
18. Through-hole in handle
19. Continuous through-hole in handle and connecting element
20. Adjustment element
21. Biobag
22. Baby
23. Open end of biobag
24. Silicone ring
25. Cord (“purse string suture”)
26. Tube connected to biobag
27. Head of baby
28. Biobag handle
29. Loop for receiving frame member
30. Attachment point for cord (×4)
31. Cord (×4)
32. Balloon
33. Pocket for receiving distal end of frame member
34. Folded portion of biobag
35. Cervix
X=Longitudinal axis
I=Insertion direction
R=Radial direction
T=Transition position
Dₘₐₓ=Max. diameter of opening between distal ends of frame members

1-29. (canceled)

30. A foetal extraction device, comprising:
a frame configured to hold a biobag inside the frame, the biobag configured to receive a baby, the frame having a distal end configured to be inserted into a vagina, a longitudinal axis aligned with an insertion direction, and a radial direction, the frame comprising frame members having distal ends configured to be inserted into the vagina and proximal ends attached to a connecting element, the frame members extending away from the connecting element in the insertion direction and being circumferentially spaced-apart around the longitudinal axis of the frame, wherein the frame members are expandable in the radial direction, and wherein a radial position or a radial orientation of the frame members is adjustable relative to the connecting element.

31. The foetal extraction device according to claim 30, wherein, when viewed along the longitudinal axis, the frame members include a first frame member portion and a second frame member portion, and wherein an interior side of the first frame member portion is concave and an interior side of the second frame member portion is convex.

32. The foetal extraction device according to claim 31, wherein the frame has a frame length L measured from the proximal ends to the distal ends of the frame members and the first concave frame member portion transitions into the second convex frame member portion at a transition position located at about 0.6 L to 0.8 L measured from the proximal ends of the frame members.

33. The foetal extraction device according to claim 30, wherein the frame members have an S-shape.

34. The foetal extraction device according to claim 30, wherein the frame members are circumferentially spaced-apart around the longitudinal axis of the frame at regular intervals.

35. The foetal extraction device according to claim 30, wherein each of the frame members comprises a group of individual, parallel frame elements extending away from the connecting element in the insertion direction.

36. The foetal extraction device according to claim 30, wherein the distal ends of the frame members include rounded knobs.

37. The foetal extraction device according to claim 30, wherein the connecting element comprises a through-hole extending through the connecting element along the longitudinal axis.

38. The foetal extraction device according to claim 30, wherein, when the frame members are arranged in a fully expanded state, a maximum diameter of an opening radially enclosed between the distal ends of the frame members is between about 6 cm and 10 cm.

39. The foetal extraction device according to claim 30, wherein the frame has a frame length L measured from the proximal ends to the distal ends of the frame members between about 30 cm and 50 cm.

40. The foetal extraction device according to claim 30, wherein at least one of the frame members has an elliptical cross-section, and wherein a short axis of the elliptical cross-section is aligned with the radial direction.

41. The foetal extraction device according to claim 30, wherein, in the radial direction, a bending stiffness at the proximal end of at least one of the frame members is greater than a bending stiffness at the distal end of the at least one of the frame members.

42. The foetal extraction device according to claim 30, further comprising a handle extending along the longitudinal axis, the handle having a proximal end and a distal end connected to the connecting element.

43. The foetal extraction device according to claim 42, wherein the handle comprises a through-hole extending through the handle along the longitudinal axis.

44. The foetal extraction device according to claim 30, further comprising an adjustment element slideably arranged over the frame members along the longitudinal axis, the adjustment element configured to adjust the radial position or the radial orientation of the frame members when sliding the adjustment element over the frame members along the longitudinal axis.

45. The foetal extraction device according to claim 44, wherein the adjustment element is a tubular element.

46. The foetal extraction device according to claim 30, further comprising a biobag disposed inside the frame for capturing a baby during delivery, wherein an open end of the biobag is positioned proximal the distal ends of the frame members.

47. The foetal extraction device according to claim 46, further comprising a vacuum ring positioned circumferentially around the open end of the biobag.

48. The foetal extraction device according to claim 46, further comprising a cord positioned circumferentially around the open end of the biobag configured to close the open end of the biobag.

49. The foetal extraction device according to claim 46, wherein the biobag further comprises at least one tube configured to supply at least one fluid to an interior of the biobag.

50. The foetal extraction device according to claim 46, further comprising at least one sensor positioned on an exterior surface of the biobag.

51. A biobag configured to receive a baby during delivery, the biobag configured to be positioned inside a foetal extraction device, the foetal extraction device comprising:
a frame configured to hold the biobag inside the frame, the frame having a distal end configured to be inserted into a vagina, a longitudinal axis aligned with an insertion direction, and a radial direction, the frame comprising frame members having distal ends configured to be inserted into the vagina and proximal ends attached to a connecting element, the frame members extending away from the connecting element in the insertion direction and being circumferentially spaced-apart around the longitudinal axis of the frame, wherein the frame members are expandable in the radial direction, and wherein a radial position or a radial orientation of the frame members is adjustable relative to the connecting element; and
wherein an open end of the biobag is positioned proximal the distal ends of the frame members.

52. The biobag according to claim 51, further comprising a vacuum ring positioned circumferentially around the open end of the biobag.

53. The biobag according to claim 51, further comprising a cord positioned circumferentially around the open end of the biobag configured to close the open end of the biobag.

54. The biobag according to claim 51, wherein the biobag further comprises at least one tube configured to supply at least one fluid to an interior of the biobag.

55. The biobag according to claim 51, further comprising at least one sensor positioned on an exterior surface of the biobag.

56. A foetal extraction device kit-of-parts, comprising:
a foetal extraction device comprising a frame having a distal end configured to be inserted into a vagina, a longitudinal axis aligned with an insertion direction, and a radial direction, the frame comprising frame members having distal ends configured to be inserted into the vagina and proximal ends attached to a connecting element, the frame members extending away from the connecting element in the insertion direction and being circumferentially spaced-apart around the longitudinal axis of the frame, wherein the frame members are expandable in the radial direction, and wherein a radial position or a radial orientation of the frame members is adjustable relative to the connecting element;
a handle configured to extend along the longitudinal axis, the handle having a proximal end and a distal end connectable to the connecting element;
an adjustment element configured to be slideably positioned over the plurality of frame members along the longitudinal axis, the adjustment element configured to adjust the radial position or the radial orientation of the frame members when sliding the adjustment element over the frame members along the longitudinal axis; and
a biobag configured to be disposed in the frame, the biobag configured to receive a baby during delivery, and wherein an open end of the biobag is configured to be positioned proximal the distal ends of the frame members.
